(19) United States
(12) Patent Application Publication
(10) Pub. No.: US 2025/0366978 A1
(43) Pub. Date: Dec. 4, 2025

(54) DEVICES AND METHODS OF MAKING AND USE THEREOF

(71) Applicant: THE METHODIST HOSPITAL, Houston, TX (US)

(72) Inventor: Aldona SPIEGEL, Houston, TX (US)

(21) Appl. No.: 18/875,376

(22) PCT Filed: Jun. 16, 2023
(86) PCT No.: PCT/US2023/025558

§ 371 Date: Dec. 16, 2024

Related U.S. Application Data
(60) Provisional application No. 63/352,744, filed on Jun. 16, 2022.

(51) Int. Cl. A61F 2/12; A61L 27/18; A61L 27/54; A61L 27/56; B33Y 80/00

(52) U.S. Cl. CPC A61F 2/12; A61L 27/18; A61L 27/54; A61L 27/56; B33Y 80/00; A61F 2240/002; A61F 2250/0018; A61F 2250/0067; A61L 2430/04

(57) ABSTRACT

Disclosed herein are devices and methods of making and use thereof.

### CROSS-REFERENCE TO RELATED APPLICATIONS

This application claims the benefit of priority to U.S. Provisional Application No. 63/352,744 filed Jun. 16, 2022, which hereby incorporated herein by reference in its entirety.

### BACKGROUND

Breast cancer is a complex disease that requires comprehensive treatment extending beyond the initial attainment of “Cancer-Free” status. Many breast cancer patients undergo mastectomy or lumpectomy procedures as part of their treatment, which often leads to significant anxiety and depression. In order to alleviate these emotional challenges, breast reconstruction has proven beneficial. However, the current state of breast reconstruction is far from perfect.

Traditionally, silicone breast implants have been widely used for reconstruction. Among the options available, there are textured shaped implants designed to maintain their shape through a velcro-like texturing effect, preventing rotation. In contrast, round smooth implants, due to their smooth surface, may rotate freely and therefore need to maintain a round shape. However, recent advancements have raised concerns regarding the safety of textured implants.

In response to the growing concerns, the U.S. Food and Drug Administration (FDA) has implemented regulatory measures to address the safety issues associated with textured implants. Notably, the FDA has issued a black box warning, specifically emphasizing the potential risks related to textured implants, particularly in relation to a rare form of BII-ALCL (anaplastic large-cell lymphoma). Consequently, only round smooth implants are currently approved for clinical use, as textured implants have been linked to these complications. This regulatory limitation necessitates the exploration of alternative methods to provide adequate support for the tissue and/or implant, while also enabling the achievement of a more natural tear drop breast shape.

In light of these safety concerns, it is crucial to develop innovative approaches that can overcome the limitations of textured implants. Strategic compression and/or elasticity offer promising avenues to address this challenge. By incorporating these techniques, alternative methods can be devised to support the tissue and/or implant effectively, while also allowing for the desired tear drop breast shape

Current solutions for providing support to an underlying organ and/or implant include ADM (acellular dermal matrix) and fabric/2D mesh structures, all of which suffer from various limitations.

With current technologies and techniques needing improvement, new methods and devices are necessary. The devices and methods discussed herein address these and other needs.

### SUMMARY

In accordance with the purposes of the disclosed devices and methods as embodied and broadly described herein, the disclosed subject matter relates to devices and methods of making and use thereof.

Additional advantages of the disclosed devices and methods will be set forth in part in the description which follows, and in part will be obvious from the description. The advantages of the disclosed devices and methods will be realized and attained by means of the elements and combinations particularly pointed out in the appended claims. It is to be understood that both the foregoing general description and the following detailed description are exemplary and explanatory only and are not restrictive of the disclosed systems and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1. AP frontal view with legend.

FIG. 2. AP frontal view.

FIG. 3. Bottom up view (worm's eye).

FIG. 4. Lateral view.

FIG. 5. Oblique Inferior view.

FIG. 6. Oblique inferior view (zoomed out).

FIG. 7. Superior view (bird's eye).

FIG. 8. Oblique view lateral.

FIG. 9. Oblique view medial.

FIG. 10. Additional design

FIG. 11. Diagram of nonauxetic (honeycomb) structure deformation mechanism.

FIG. 12. Diagram of auxetic (reentrant) structure deformation mechanism.

FIG. 13. Deformation of auxetic and conventional materials.

FIG. 14a-FIG. 14b. Cell deformation by inclined cell member bending (FIG. 14a) loaded in X-direction, (FIG. 14b) loaded in Y direction.

FIG. 15. Rigid rectangles connected together at their vertices through hinges and deforming by rotating.

FIG. 16. Anatomical image of subject with Fibonacci curve overlaid.

FIG. 17. Schematic view of an example device as disclosed herein according to one implementation.

FIG. 18. Schematic cut away side view of an example device in use within a breast of a subject as disclosed herein according to one implementation.

FIG. 19. Schematic view of an example device as disclosed herein according to one implementation.

FIG. 20. Schematic view of an example device as disclosed herein according to one implementation.

FIG. 21. Schematic view of an example device as disclosed herein according to one implementation.

FIG. 22. Schematic cut away side view of an example device in use within a breast of a subject as disclosed herein according to one implementation.

FIG. 23. Schematic cut away side view of an example device in use within a breast of a subject as disclosed herein according to one implementation.

FIG. 24. Schematic view of an example device as disclosed herein according to one implementation.

FIG. 25. Schematic view of an example device as disclosed herein according to one implementation.

FIG. 26. Schematic cut away side view of an example device in use within a breast of a subject as disclosed herein according to one implementation.

FIG. 27. Schematic cut away side view of an example device in use within a breast of a subject as disclosed herein according to one implementation.

FIG. 28. Schematic cut away side view of an example device in use within a breast of a subject as disclosed herein according to one implementation.

FIG. 29. Schematic view of an example device as disclosed herein according to one implementation.

FIG. 30. Schematic view of an example device as disclosed herein according to one implementation.

FIG. 31. Schematic view of an example device as disclosed herein according to one implementation.

FIG. 32. Schematic view of an example device as disclosed herein according to one implementation.

FIG. 33. Schematic view of an example device as disclosed herein according to one implementation.

FIG. 34. Schematic view of an example device as disclosed herein according to one implementation.

FIG. 35. Schematic view of an example device as disclosed herein according to one implementation.

### DETAILED DESCRIPTION

The devices and methods described herein may be understood more readily by reference to the following detailed description of specific aspects of the disclosed subject matter and the Examples included therein.

Before the present devices and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification, the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Values can be expressed herein as an “average” value. “Average” generally refers to the statistical mean value.

By “substantially” is meant within 5%, e.g., within 4%, 3%, 2%, or 1%.

“Exemplary” means “an example of” and is not intended to convey an indication of a preferred or ideal embodiment. “Such as” is not used in a restrictive sense, but for explanatory purposes.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

“Biocompatible” and “biologically compatible”, as used herein, generally refer to compounds and/or compositions that are, along with any metabolites or degradation products thereof, generally non-toxic to normal cells and tissues, and which do not cause any significant adverse effects to normal cells and tissues when cells and tissues are incubated (e.g., cultured) in their presence.

The term “biodegradable” or “bioresorbable” as used herein refers to a material or substance wherein physical dissolution and/or chemical degradation is effected under physiological conditions.

As used herein, “antimicrobial” refers to the ability to treat or control (e.g., reduce, prevent, treat, or eliminate) the growth of a microbe at any concentration. Similarly, the terms “antibacterial,” “antifungal,” and “antiviral” refer to the ability to treat or control the growth of bacteria, fungi, and viruses at any concentration, respectively.

As used herein, “reduce” or other forms of the word, such as “reducing” or “reduction,” refers to lowering of an event or characteristic (e.g., microbe population/infection). It is understood that the reduction is typically in relation to some standard or expected value. For example, “reducing microbial infection” means reducing the spread of a microbial infection relative to a standard or a control.

As used herein, “prevent” or other forms of the word, such as “preventing” or “prevention,” refers to stopping a particular event or characteristic, stabilizing or delaying the development or progression of a particular event or characteristic, or minimizing the chances that a particular event or characteristic will occur. “Prevent” does not require comparison to a control as it is typically more absolute than, for example, “reduce.” As used herein, something could be reduced but not prevented, but something that is reduced could also be prevented. Likewise, something could be prevented but not reduced, but something that is prevented could also be reduced.

As used herein, “treat” or other forms of the word, such as “treated” or “treatment,” refers to administration of a composition or performing a method in order to reduce, prevent, inhibit, or eliminate a particular characteristic or event (e.g., microbe growth or survival). The term “control” is used synonymously with the term “treat.”

The term “anticancer” refers to the ability to treat or control cellular proliferation and/or tumor growth at any concentration.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, “molecular weight” refers to the number average molecular weight as measured by ¹H NMR spectroscopy, unless indicated otherwise.

### Devices

Disclosed herein are devices configured to be inserted into an anatomical location of a subject. In some examples, the device is a mesh with a plurality of zones having different elastic properties, each zone having a composition and a pattern, wherein the different elastic properties are based on the composition, the pattern, or combination thereof. In some examples, the composition of each of the plurality of zones comprises an isotropic material, an anisotropic material, an auxetic material, a non-auxetic material, or a combination thereof. In some examples, the pattern of each of the plurality of zones comprises an auxetic pattern, a non-auxetic pattern, or a combination thereof.

Non-auxetic materials and/or patterns have a positive Poisson's ratio and become thinner in cross section when stretched. The transverse contraction strain to longitudinal extension strain in the direction of the stretching is positive.

Examples of a non-auxetic pattern include, but are not limited to, lattice structures based on a diamond, cube, truncated cube, rhombic dodecahedron, and truncated cuboctahedron unit cells.

Auxetic materials and/or patterns have a negative Poisson's ratio; when stretched, they become thicker perpendicular to the applied force. This occurs due to their particular internal structure and the way this deforms when uniaxially loaded. Auxetics can be single molecules, crystals, or a particular structure of macroscopic matter. Examples of auxetics include, but are not limited to re-entrant structures, chiral structures, rotational (semi-)rigid structures, crumpled and perforated sheet models, and miscellaneous structure which includes arbitrary geometries (e.g., egg rack model, tethered nodule, hexa-truss model, and origami structure).

Re-entrant structures can, for example, include the re-entrant corner and re-entrant angle in an irregular polygon. The re-entrant angle in an irregular polygon is an interior angle that is greater than 180°, as seen in the ribs of the “bow-tie” honeycomb (FIG. 12). Re-entrant structures can be formed by hexagonal face cells, which have the edges protruding outwardly. Along with re-alignment (hinging), deflection and axial deformation (stretching) of cell ribs can also be responsible for the deformation of re-entrant structures and auxetic behavior.

The plurality of zones can, for example, each have a different composition, a different pattern, or a combination thereof. In some examples, the plurality of zones have the same composition and different patterns, such that the different properties are based on the pattern of each zone. In some examples, the plurality of zones each have a different composition and a different pattern.

In some examples, the mesh comprises a fat grafting zone having little to no elasticity. The fat grating zone can, for example, comprises adipose tissue, such as autologous adipose tissue.

In some examples, the mesh comprises a support zone, the support zone being elastic along one direction. The support zone can, for example, have a non-auxetic pattern. In some examples, the support zone comprises an anisotropic material, a non-auxetic material, or a combination thereof.

In some examples, the mesh comprises an elastic zone, the elastic zone having a high amount of elasticity in multiple directions. The elastic zone can, for example, have an auxetic pattern. In some examples, the elastic zone can comprise an isotropic material, an auxetic material, or a combination thereof.

In some examples, the mesh comprises a transition zone, the transition zone being more elastic than the support zone and less elastic than the elastic zone.

In some examples, the mesh comprises a skin defect zone, the skin defect zone being aligned with skin-growth promoting stroma such that the skin defect zone facilitates epithelization and skin coverage when implanted in the subject.

Each of the plurality of zones can comprise any suitable composition. The composition of each of the plurality of zones can, for example, independently comprise a polymer such as a bioresorbable polymer, collagen, adipose cellular stromal matrix, adipose tissue, or a combination thereof.

In some examples, one or more of the plurality of zones can each independently comprise poly(ethylene glycol) diacrylate (PEGDA), poly(ethylene glycol) dimethacrylate (PEGDMA), poly(ethylene glycol) diacrylamide (PEGDAAm), gelatin methacrylate (GelMA), collagen methacrylate, silk methacrylate, hyaluronic acid methacrylate, chondroitin sulfate methacrylate, elastin methacrylate, cellulose acrylate, dextran methacrylate, heparin methacrylate, NIPAAm methacrylate, Chitosan methacrylate, polyethylene glycol norbornene, polyethylene glycol dithiol, thiolated gelatin, thiolated chitosan, thiolated silk, silk (e.g., from silkworms and/or spiders), PEG based peptide conjugates, cell-adhesive poly(ethylene glycol), MMP-sensitive poly(ethylene glycol), PEGylated fibrinogen, aliphatic poly-isocyanate, poly-aliphatic isocyanates, poly-4-hudroxybutyrate, poly(l-lactide) (PLLA), bioceramic particles, L-lactide (LLA), sub-dermal explant comprising polycaprolactone (PCL), polyurethane, poly(D) lactide, poly(lactic-co-glycolic) acid, poly(α-hydroxy acids), cross-linked polyester hydrogels, poly(orthoesters), polyanhydrides, or a combination thereof. In some examples, the one or more of the plurality of zones can each independently comprise aliphatic poly-isocyanate, poly-aliphatic isocyanates, poly-4-hudroxybutyrate, poly(l-lactide) (PLLA), bioceramic particles, L-lactide (LLA), sub-dermal explant comprising polycaprolactone (PCL), polyurethane, poly(D) lactide, poly(lactic-co-glycolic) acid, poly(α-hydroxy acids), cross-linked polyester hydrogels, poly(orthoesters), polyanhydrides, or a combination thereof.

In some examples, one or more of the plurality of zones can each independently comprise a polyester, such as poly(glycerol-dodecanoate) (PGD).

In some examples, one or more of the plurality of zones can each independently comprise a poly(ether-ester). In some examples, one or more of the plurality of zones can each independently comprise polydioxanone (PDO). In some examples, each of the plurality of zones comprises polydioxanone (PDO).

In some examples, one or more of the plurality of zones can each independently comprise a polyolefin, such as polypropylene.

In some examples, one or more of the plurality of zones can each independently comprise polyglycolic acid (PGA).

In some examples, one or more of the plurality of zones can each independently comprise polyglycolic acid (PGA) or a copolymer thereof, polylactic acid (PLA) or a copolymer thereof, polycaprolactone (PCL) or a copolymer thereof, or a combination thereof.

In some examples, one or more of the plurality of zones can each independently comprise a bioink. Examples of bioinks include, but are not limited to, alginate-based bioinks, gelatin-based bioinks (e.g., GelMA—Gelatin Methacryloyl), collagen-based bioinks (e.g., Type I collagen, Type II collagen), fibrin-based bioinks, chitosan-based bioinks, hyaluronic acid-based bioinks, Matrigel-based bioinks, silk fibroin-based bioinks, cellulose-based bioinks, polyethylene glycol-based bioinks (PEG-based bioinks), polycaprolactone-based bioinks (PCL-based bioinks), poly(lactic-co-glycolic acid)-based bioinks (PLGA-based bioinks), polyvinyl alcohol-based bioinks (PVA-based bioinks), polyurethane-based bioinks, polydopamine-based bioinks, polypeptide-based bioinks, agarose-based bioinks, carboxymethyl cellulose-based bioinks, sodium alginate-gelatin-based bioinks, polyethylene oxide-based bioinks (PEO-based bioinks), and combinations thereof.

In some examples, one or more of the plurality of zones can each independently comprise collagen.

In some examples, the composition of one of more of the plurality of zones can each independently be is porous.

In some examples, the device further comprises a therapeutic agent dispersed within the composition of one or more of the plurality of zones. In some examples, the therapeutic agent is dispersed inhomogeneously throughout the zone(s) and/or device (e.g., randomly, along a concentration gradient, only in certain zones, etc.). In some examples, the therapeutic agent is dispersed substantially homogeneously throughout the zone(s) and/or device.

The therapeutic agent can, for example, comprise an anticancer agent, anti-inflammatory agent, analgesic agent, antimicrobial agent, or a combination thereof. As used herein, antimicrobials include, for example, antibacterials, antifungals, and antivirals.

Examples of antimicrobial agents include, but are not limited to, alexidine, asphodelin A, atromentin, auranthine, austrocortilutein, austrocortirubin, azerizin, chlorbisan, chloroxine, cidex, cinoxacin, citreorosein, copper usnate, cupiennin, curvularin, DBNPA, dehydrocurvularin, desoxyfructo-serotonin, dichloroisocyanuric acid, elaiomycin, holtfreter's solution, malettinin, naphthomycin, neutrolin, niphimycin, nitrocefin, oxadiazoles, paenibacterin, proclin, ritiometan, ritipenem, silicone quaternary amine, stylisin, taurolidine, tirandamycin, trichloroisocyanuric acid, triclocarban, and combinations thereof.

Examples of antibacterials include, but are not limited to, acetoxycycloheximide, aciduliprofundum, actaplanin, actinorhodin, alazopeptin, albomycin, allicin, allistatin, allyl isothiocyanate, ambazone, aminocoumarin, aminoglycosides, 4-aminosalicylic acid, ampicillin, ansamycin, anthramycin, antimycin A, aphidicolin, aplasmomycin, archaeocin, arenicin, arsphenamine, arylomycin A2, ascofuranone, aspergillic acid, avenanthramide, avibactam, azelaic acid, bafilomycin, bambermycin, beauvericin, benzoyl peroxide, blasticidin S, bottromycin, brilacidin, caprazamycin, carbomycin, cathelicidin, cephalosporins, ceragenin, chartreusin, chromomycin A3, citromycin, clindamycin, clofazimine, clofoctol, clorobiocin, coprinol, coumermycin A1, cyclic lipopeptides, cycloheximide, cycloserine, dalfopristin, dapsone, daptomycin, debromomarinone, 17-dimethylaminoethylamino-17-demethoxygeldanamycin, echinomycin, endiandric acid C, enediyne, enviomycin, eravacycline, erythromycin, esperamicin, etamycin, ethambutol, ethionamide, (6S)-6-fluoroshikimic acid, fosfomycin, fosmidomycin, friulimicin, furazolidone, furonazide, fusidic acid, geldanamycin, gentamycin, gepotidacin, glycyclclines, glycyrrhizol, gramicidin S, guanacastepene A, hachimycin, halocyamine, hedamycin, helquinoline, herbimycin, hexamethylenetetramine, hitachimycin, hydramacin-1, isoniazid, kanamycin, katanosin, kedarcidin, kendomycin, kettapeptin, kidamycin, lactivicin, lactocillin, landomycin, landomycinone, lasalocid, lenapenem, leptomycin, lincosamides, linopristin, lipiarmycins, macbecin, macrolides, macromomycin B, maduropeptin, mannopeptimycin glycopeptide, marinone, meclocycline, melafix, methylenomycin A, methylenomycin B, monensin, moromycin, mupirocin, mycosubtilin, myriocin, myxopyronin, naphthomycin A, narasin, neocarzinostatin, neopluramycin, neosalvarsan, neothramycin, netropsin, nifuroxazide, nifurquinazol, nigericin, nitrofural, nitrofurantoin, nocathiacin I, novobiocin, omadacycline, oxacephem, oxazolidinones, penicillins, peptaibol, phytoalexin, plantazolicin, platensimycin, plectasin, pluramycin A, polymixins, polyoxins, pristinamycin, pristinamycin IA, promin, prothionamide, pulvinone, puromycin, pyocyanase, pyocyanin, pyrenocine, questiomycin A, quinolones, quinupristin, ramoplanin, raphanin, resistome, reuterin, rifalazil, rifamycins, ristocetin, roseophilin, salinomycin, salinosporamide A, saptomycin, saquayamycin, seraticin, sideromycin, sodium sulfacetamide, solasulfone, solithromycin, sparassol, spectinomycin, staurosporine, streptazolin, streptogramin, streptogramin B, streptolydigin, streptonigrin, styelin A, sulfonamides, surfactin, surotomycin, tachyplesin, taksta, tanespimycin, telavancin, tetracyclines, thioacetazone, thiocarlide, thiolutin, thiostrepton, tobramycin, trichostatin A, triclosan, trimethoprim, trimethoprim, tunicamycin, tyrocidine, urauchimycin, validamycin, viridicatumtoxin B, vulgamycin, xanthomycin A, xibornol, amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomycin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, vancomycin, and combinations thereof.

Examples of antifungals include, but are not limited to, abafungin, acibenzolar, acibenzolar-S-methyl, acrisorcin, allicin, aminocandin, amorolfine, amphotericin B, anidulafungin, azoxystrobin, bacillomycin, Bacillus pumilus, barium borate, benomyl, binapacryl, boric acid, bromine monochloride, bromochlorosalicylanilide, bupirimate, butenafine, candicidin, caprylic acid, captafol, captan, carbendazim, caspofungin, cerulenin, chloranil, chlormidazole, chlorophetanol, chlorothalonil, chloroxylenol, chromated copper arsenate, ciclopirox, cilofungin, cinnamaldehyde, clioquinol, copper(I) cyanide, copper(II) arsenate, cruentaren, cycloheximide, davicil, dehydroacetic acid, dicarboximide fungicides, dichlofluanid, dimazole, diphenylamine, echinocandin, echinocandin B, epoxiconazole, ethonam, falcarindiol, falcarinol, famoxadone, fenamidone, fenarimol, fenpropimorph, fentin acetate, fenticlor, filipin, fluazinam, fluopicolide, flusilazole, fluxapyroxad, fuberidazole, griseofulvin, halicylindramide, haloprogin, hamycin, hexachlorobenzene, hexachlorocyclohexa-2,5-dien-1-one, 5-hydroxy-2(5H)-furanone, iprodione, lime sulfur, mancozeb, maneb, melafix, metalaxyl, metam sodium, methylisothiazolone, methylparaben, micafungin, miltefosine, monosodium methyl arsenate, mycobacillin, myclobutanil, natamycin, beta-nitrostyrene, nystatin, paclobutrazol, papulacandin B, parietin, pecilocin, pencycuron, pentamidine, pentachloronitrobenzene, pentachlorophenol, perimycin, 2-phenylphenol, polyene antimycotic, propamocarb, propiconazole, pterulone, ptilomycalin A, pyrazophos, pyrimethanil, pyrrolnitrin, selenium disulfide, sparassol, strobilurin, sulbentine, tavaborole, tebuconazole, terbinafine, theonellamide F, thymol, tiabendazole, ticlatone, tolciclate, tolnaftate, triadimefon, triamiphos, tribromometacresol, 2,4,6-tribromophenol, tributyltin oxide, triclocarban, triclosan, tridemorph, trimetrexate, undecylenic acid, validamycin, venturicidin, vinclozolin, vinyldithiin, vusion, xanthene, zinc borate, zinc pyrithione, zineb, ziram, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, amphotericin b., and combinations thereof.

Examples of antivirals include, but are not limited to, afovirsen, alisporivir, angustific acid, angustifodilactone, alovudine, beclabuvir, 2,3-bis(acetylmercaptomethyl)quinoxaline, brincidofovir, dasabuvir, docosanol, fialuridine, ibacitabine, imiquimod, inosine, inosine pranobex, interferon, metisazone, miltefosine, neokadsuranin, neotripterifordin, ombitasvir, oragen, oseltamivir, pegylated interferon, podophyllotoxin, radalbuvir, semapimod, tecovirimat, telbivudine, theaflavin, tilorone, triptofordin C-2, variecolol, ZMapp, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, zidovudine. and combinations thereof.

In some examples, the therapeutic agent can comprise an anticancer agent. In some examples, the therapeutic agent comprises a chemotherapeutic agent, an immunotherapeutic agent, or a combination thereof.

In some examples, the therapeutic agent can comprise a chemotherapeutic agent. Chemotherapy is the treatment of cancer with one or more cytotoxic anti-neoplastic drugs (e.g., chemotherapeutic agents) as part of a standardized regimen. Chemotherapy may be given with a curative intent or it may aim to prolong life or to palliate symptoms. In some cases, it can be used in conjunction with other cancer treatments, such as radiation therapy, surgery, hyperthermia therapy, or a combination thereof. Examples of chemotherapeutic agents include, but are not limited to, 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, LCR, FAM-HYD-1, Marizomib (NPI-0052), Lenalidomide, Carfilzomib, Panobinostat, Quisinostat, Selinexor, Oprozomib, and combinations thereof. The anticancer agent can also include biopharmaceuticals such as, for example, antibodies.

Examples of suitable immunotherapeutic agents include, but are not limited to, alemtuzumab, cetuximab (ERBITUX), gemtuzumab, iodine 131 tositumomab, rituximab, trastuzamab (HERCEPTIN), and combinations thereof.

In some examples, the therapeutic agent can comprise an anti-inflammatory agent, such as steroidal and/or non-steroidal anti-inflammatory agents. Examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Examples of non-steroidal anti-inflammatory drugs include acetaminophen, aspirin, ibuprofen, naproxen, Celebrex, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

In some examples, the therapeutic agent can comprise an analgesic. Examples of analgesics include, but are not limited to, 1-Iodomorphine; 3-Hydroxymorphinan; 4-Methylpregabalin; A-366,833; ABT-202; Aceburic acid; Acefurtiamine; Acetaminosalol; Acetyldihydrocodeine; Acetylmethadol; Adrenorphin; Alazocine; Algifen; Alimadol; Alletorphine; Alphacetylmethadol; Alphamethadol; Amidorphin; Aminophenazone; Ampyrone; Amrutanjan (balm); Anacin; Anadin; Analgecine; Anazocine; Anileridine; Anilopam; Anodyne; Askit Powders; Aspergum; Aspirin; Axomadol; AZD0328; BC Powder; Befiradol; Benorilate; Betacetylmethadol; Betahydroxyfentanyl; Betamethadol; Bicifadine; Biphalin; Brorphine; Bucetin; Bucinnazine; Butalbital; Butinazocine; Butonitazine; Butorphanol; Cannabidiol; Carbazocine; Cebranopadol; Chlorodyne; Chlorproethazine; Cinchophen; Cogazocine; Conolidine; Conorfone; CR-4056; CR665; Dasolampanel; Deltorphin; Deltorphin I; DepoDur; Desmetramadol; Desomorphine; Dezocine; Diacetylnalorphine; Dichloralphenazone; Difenamizole; Dimenoxadol; Dimepheptanol; Dimethylheptylpyran; Dinalbuphine sebacate; Dipipanone; Diproqualone; Dipyrocetyl; Dosulepin; DSP-2230; Embutramide; Enkephalinase inhibitor; Epibatidine; Epiboxidine; Eptazocine; Esterom; Etazocine; Ethylketazocine; Etodesnitazene; Etonitazepipne; Etonitazepyne; Etorphine; Famotidine; Faxeladol; Fedotozine; Fentanyl; Filenadol; Flumexadol; Flumizole; Fluproquazone; Frakefamide; Funapide; Gabapentin; Gabapentin enacarbil; Gabapentinoid; Glafenine; Homofentanyl; Homprenorphine; Ibazocine; Ibuprofen; Incarvillateine; Indantadol; Isomethadone; Isotonitazene; Isovaline; Kavalactone; Kelatorphan; Ketamine; Ketobemidone; Ketorfanol; Ketorolac; Lactucarium; Leconotide; Levallorphan; Levomepromazine; Levomethadone; Lufuradom; Magnesium alicylate; Mavatrep; Meconopsis horridula; Menabitan; Menthol; Menthoxypropanediol; Meprobamate; Meseclazone; Metacetamol; Metamizole; Methoxyflurane; Metkefamide; Metodesnitazene; Metonitazene; Mexiletine; Migraleve; Mirogabalin; Mitragyna speciosa; Moffett's solution; Moramide intermediate; Morpheridine; Morphiceptin; Morphine; Moxazocine; MP-2001; N-2′-Indolylnaltrexamine; Nabilone; Nafoxadol; Nalbuphine; Nalmexone; Naproxen; Nefopam; Nexeridine; NFEPP; Nimesulide; Noracymethadol; Norlevorphanol; Normethadone; Norpipanone; NS-11394; Oliceridine; Opiorphin; Opiranserin; Opium; Otenaproxesul; Oxilorphan; Paracetamol; Pethidine; PF-05089771; Phenacetin; Phenazone; derivatives thereof; and combinations thereof.

In some examples, the therapeutic agent can comprise an analgesic, such as an opioid. Examples of opioids include, but are not limited to, (α/β)-Meprodine; (α/β)-Prodine; 1-(4-Nitrophenylethyl)piperidylidene-2-(4-chlorophenyl)sulfonamide (W-18); 14-Cinnamoyloxycodeinone; 14-Ethoxymetopon; 14-Hydroxydihydrocodeine; 14-Hydroxymorphine; 14-Methoxymetopon; 14-Phenylpropoxymetopon; 18,19-Dehydrobuprenorphine (HS-599); 18-Methoxycoronaridine; 1-Bromocodeine; 1-Chlorocodeine; 1-Iodomorphine Codeine-6-glucuronide; 1-Nitrocodeine; 2,4-Dinitrophenylmorphine; 3-(3-Methoxyphenyl)-3-ethoxycarbonyltropane; 3-(dimethylamino)-2,2-dimethyl-1-phenylpropan-1-one; 3,14-Diacetyloxymorphone; 3,6-Dibutanoylmorphine; 3-Acetyloxymorphone; 3-Allylfentanyl; 3-Hydroxymorphinan; 3-Methylfentanyl; 3-Methylthiofentanyl; 3-Monoacetylmorphine; 4-Chlorophenylpyridomorphinan; 4-Fluoropethidine; 4-Phenylfentanyl; 5,6-Dihydronorsalutaridine; 5,9 alpha-diethyl-2-hydroxybenzomorphan (5,9-DEHB); 6-Acetyldihydromorphine; 6-Keto Nalbuphine; 6-Methyldihydromorphine; 6-Methylenedihydrodesoxymorphine; 6-Monoacetylcodeine; 6-Monoacetylmorphine; 6-Nicotinoyldihydromorphine; 7-Acetoxymitragynine; 7-Hydroxymitragynine; 7-PET; 7-Spiroindanyloxymorphone; 8,14-Dihydroxydihydromorphinone; 8-Carboxamidocyclazocine (8-CAC); Acetorphine; Acetoxyketobemidone; Acetylcodone; Acetyldihydrocodeine; Acetylmethadol; Acetylmorphone; Acetylpropionylmorphine; AD-1211; ADL-5859; AH-7921; Aknadinine; Akuammidine; Akuammine; Alazocine; Alfentanil; Alimadol; Alletorphine (N-allyl-noretorphine); Allylnorpethidine; Allylprodine; Alphaacetylmethadol; Alphamethadol; Alvimopan; Amentoflavone; Anazocine; Anileridine; Anilopam+HCl; Asimadoline; Axomadol; Azaprocin; AZD-2327; Azidomorphine; BDPC; Benzethidine; Benzhydrocodone; Benzylfentanyl; Benzylmorphine; Betacetylmethadol; Betamethadol; Bezitramide; Bisnortilidine; Bremazocine; Brifentanil; BRL-52537; Bromadol; Bromadoline; Bromocodide; Bromoisopropropyldihydromorphinone; Bromomorphide; BU-48; Buprenorphine; Buprenorphine-3-glucuronide; Butinazocine; Butorphanol; Butyrfentanyl; BW373U86; Carbazocine; Carfentanil; Carperidine; Cephakicine; Cephasamine; Chlornaltrexamine; Chlorodihydrocodide; Chloromorphide; Chloroxymorphamine; Ciprefadol; Ciramadol; Clonitazene; Codeine; Codeine methylbromide; Codeine-N-oxide; Codeine-N-oxide (genocodeine); Codeinone; Codide; Codoxime; Cogazocine; Conorfone (codorphone); Coronaridine; Cyclazocine; Cyclorphan; Cyprenorphine; Cyprodime; Cyproterone acetate; Desmethylclozapine; Desmethylmoramide; Desmethylprodine (MPPP); Desocodeine Desomorphine (dihydrodesoxymorphine); Dextromethadone; Dextromoramide; Dextropropoxyphene (propoxyphene); Dezocine; Diacetyldihydromorphine (dihydroheroin, acetylmorphinol); Diampromide; Dibenzoylmorphine; Dibutyrylmorphine; Diethylthiambutene; Difenoxin; Diformylmorphine; Dihydrocodeine; Dihydrocodeine; Dihydrodesoxycodeine (desocodeine); Dihydroetorphine; Dihydroisocodeine; Dihydromorphine; Dimenoxadol; Dimepheptanol (racemethadol); Dimethylmorphine (6-O-Methylcodeine); Dimethylthiambutene; Dioxaphetyl butyrate; Diphenoxylate; Dipipanone; Dipropanoylmorphine; Doxpicomine; DPI-221; DPI-287; DPI-3290; Drotebanol; Droxypropine; Embutramide; Enadoline; Eptazocine; Eseroline; Etazocine; Ethoheptazine; Ethyldihydromorphine; Ethylketazocine; Ethylmethylthiambutene; Ethylmorphine (dionine); Etonitazene; Etorphine; Etoxeridine (carbetidine); Faxeladol; FE 200665; Fedotozine; Fenfangjine G; Fentanyl; Fluorophen; Furethidine; Gemazocine; GR-89696; Herkinorin; Heroin (diacetylmorphine); Heroin-7,8-oxide; Heterocodeine; Hodgkinsine; Homprenorphine; Hydrocodone; Hydromorphinol; Hydromorphone; Hydroxycodeine; Hydroxypethidine (bemidone); HZ-2; Ibazocine; IBNtxA; Ibogaine; IC-26; ICI-199,441; ICI-204,448; Isoaminile; Isocodeine; Isomethadol; Isomethadone; Isotonitazene; Ketamine; Ketazocine; Ketobemidone; Ketorfanol; KNT-42; Kolokol-1; Lefetamine; Levacetylmethadol; Levargorphan; Levoisomethadone; Levomethadone; Levomethorphan; Levomoramide; Levophenacylmorphan; Levopropoxyphene; Levorphanol; Lofentanil; Loperamide; LPK-26; LS-115509; Lufuradom; Matrine; MCOPPB; Menthol; Meperidine-N-oxide; Meptazinol; Metazocine; Metethoheptazine; Methadone; Metheptazine; Methorphan (racemethorphan); Methyldesorphine; Methyldihydromorphine (dihydroheterocodeine); Methyldihydromorphinone; Methylketobemidone; Metofoline; Metonitazene; Metopon; Mirfentanil; Mitragynine; Mitragynine pseudoindoxyl; Morphanol (racemorphanol); Morphenol; Morpheridine; Morphine; Morphine methylbromide; Morphine-6-glucuronide; Morphine-N-oxide; Morphine-N-oxide (genomorphine); Morphinone; Morphol; Moxazocine; MT-45; MT-7716; Myrophine; Nalbuphine; Nalbuphone; Nalfurafine; Nalorphine; Nalorphine dinicotinate; Naltrexol; N-cyclopropylmethylnoretorphine; Nepenthone; Nexeridine; Nicocodeine; Nicodicodeine; Nicomorphine; N-Methylcarfentanil; N-Methylmorphinan; NNC 63-0532; Noracymethadol; Norbuprenorphine; Norbuprenorphine-3-glucuronide; Norcodeine; Noribogaine; Norlevorphanol; Normethadone; Normorphine; Noroxymorphone; Norpipanone; Norpropoxyphene; Nortilidine; N-Phenethyl-14-ethoxymetopon; N-Phenethyl-14-ethoxymetopon; N-Phenethylnordesomorphine; N-Phenethylnormorphine; Ocfentanil; O-Desmethyltramadol; Ohmefentanyl; Opium; Oripavine; Oxilorphan; Oxpheneridine (carbamethidine); Oxycodone; Oxymorphazone; Oxymorphol; Oxymorphone; Pantopon; Papaveretum (Omnopon); Parafluorofentanyl; Pentamorphone; Pentazocine; PEPAP; Pericine; Pethidine (meperidine); Phenadone; Phenadoxone (heptazone); Phenampromide; Phenaridine; Phenazocine; Phencyclidine; Pheneridine; Phenomorphan; Phenoperidine; Pholcodine (morpholinylethylmorphine); Picenadol; Piminodine; Piperidylthiambutene; Piritramide; Prodilidine; Profadol; Proglumide; Proheptazine; Properidine (ipropethidine); Propiram; Propylketobemidone; Prosidol; Proxorphan; Pseudoakuammigine; Pseudomorphine; Pyrrolidinylthiambutene; Pyrroliphene; PZM21; Quadazocine; R-30490; R-4066; Racemoramide; RAM-378; Remifentanil; Ro-1539; Ro4-1539; Ro64-6198; Ro65-6570; RWJ-394,674; Salvinorin A; Salvinorin B ethoxymethyl ether; Salvinorin B methoxymethyl ether; Sameridine; SB-612,111; SC-17599; Semorphone; SKF-10047; SNC-80; SoRI-9409; Spiradoline; SR-16435; SR-8993; Sufentanil; TAN-67; Tannagine; Tapentadol; Tetrapon; Thebacon; Thebacon (acetyldihydrocodeinone, dihydrocodeinone enol acetate); Thebaine; Thenylfentanyl; Thevinone; Thiambutene; Thiazocine; Thienorphine; Thiobromadol (C-8813); Thiofentanyl; Tifluadom; Tilidine; Tonazocine; Tramadol; Transisocodeine; Trefentanil; Trimebutine; Trimeperidine (promedol); U-47700; U-50,488; U-69,593; Viminol; Volazocine; Zenazocine; α-Chlorocodide; α-Chloromorphide; α-hydrocodol; α-Methylacetylfentanyl; α-Methylfentanyl; α-Methylthiofentanyl; β-Chlorocodide; β-hydroxyfentanyl; β-hydroxythiofentanyl; β-Methylfentanyl; ψ-Akuammigine; derivatives thereof; and combinations thereof.

The device can, for example, be configured to support an organ and/or an implant when inserted in the subject. In some examples, the anatomical location comprises a breast of the subject. In some examples, the device is configured to support at least a portion of a breast or at least a portion of a breast implant. In some examples, the device is configured to support a breast or a breast implant.

In some examples, device is formed from a model based on a tessellation of polyhedrons. For example, the device can be formed from a computational 3D space-filling model.

In some examples, the device is not flat. For example, the device can have a three dimensional shape, such as a three-dimensional parametric teardrop shape. In some examples, the device has a two dimensional shape that can stretch to a three dimensional teardrop shape that conforms to support and/or reshape an organ and/or an implant when inserted in the subject. In some examples, the device has a three-dimensional parametric teardrop shape following the Fibonacci equation.

In some examples, device is a single piece of mesh (e.g., monolithic).

In some examples, the device is implantable in a subject. In some examples, the device is biocompatible. In some examples, the device is anatomically designed for the subject. In some examples, the anatomical location comprises at least a portion of a breast of the subject. In some examples, the anatomical location comprises a breast of the subject.

In some examples, the device is configured to be stable for an amount of time after the device is implanted in the subject. As used herein, “stable” means that 10 wt % or less (e.g., 9% or less, 8% or less, 7% or less, 6% or less, 5% or less, 4% or less, 3% or less, 2% or less, or 1% or less) of the device biodegrades over the selected time period after the device is implanted in the subject.

In some examples, the device is configured to be stable for an amount of time of 6 weeks or more after the device is implanted in the subject (e.g., 7 weeks or more, 8 weeks or more, 9 weeks or more, 10 weeks or more, 11 weeks or more, 12 weeks or more, 13 weeks or more, 14 weeks or more, 15 weeks or more, 16 weeks or more, 17 weeks or more, 18 weeks or more, 19 weeks or more, 20 weeks or more, 21 weeks or more, 22 weeks or more, 23 weeks or more, 6 months or more, 7 months or more, 8 months or more, 9 months or more, 10 months or more, 11 months or more, 12 months or more, 13 months or more, 14 months or more, 15 months or more, 16 months or more, 17 months or more, 18 months or more, 19 months or more, 20 months or more, 21 months or more, 22 months or more, 23 months or more, 24 months or more, 25months or more, 26 months or more, 27 months or more, 28 months or more, 29 months or more, 30 months or more, 31 months or more, 32 months or more, 33 months or more, 34 months or more, 35 months or more, 36 months or more, 3.5 years or more, 4 years or more, or 4.5 years or more). In some examples, the device is configured to be stable for an amount of time of 5 years or less after the device is implanted in the subject (e.g., 4.5 years or less, 4 years or less, 3.5 years or less, 36 months or less, 35 months or less, 34 months or less, 33 months or less, 32 months or less, 31 months or less, 30 months or less, 29 months or less, 28 months or less, 27 months or less, 26 months or less, 25 months or less, 24 months or less, 23 months or less, 22 months or less, 21 months or less, 20 months or less, 19 months or less, 18 months or less, 17 months or less, 16 months or less, 15 months or less, 14 months or less, 13 months or less, 12 months or less, 11 months or less, 10 months or less, 9 months or less, 8 months or less, 7 months or less, 6 months or less, 23 weeks or less, 22 weeks or less, 21 weeks or less, 20 weeks or less, 19 weeks or less, 18 weeks or less, 17 weeks or less, 16 weeks or less, 15 weeks or less, 14 weeks or less, 13 weeks or less, 12 weeks or less, 11 weeks or less, 10 weeks or less, 9 weeks or less, 8 weeks or less, or 7 weeks or less). The amount of time for which the device is configured to be stable can range from any of the minimum values described above to any of the maximum values described above. For example, the device can be configured to be stable for an amount of time of from 6 weeks to 5 years after the device is implanted in the subject (e.g., from 6 weeks to 6 months, from 6 months to 5 years, from 6 weeks to 12 weeks, from 12 weeks to 18 weeks, from 18 weeks to 6 months, from 6 months to 12 months, from 12 months to 18 months, from 18 months to 24 months, from 24 months to 30 months, from 30 months to 36 months, from 36 months to 5 years, from 7 weeks to 5 years, from 6 weeks to 4.5 years, from 7 weeks to 4.5 years, from 8 weeks to 5 years, from 10 weeks to 5 years, from 12 weeks to 5 years, from 16 weeks to 5 years, from 20 weeks to 5 years, from 6 months to 5 years, from 8 months to 5 years, from 10 months to 5 years, from 12 months to 5 years, from 18 months to 5 years, from 24 months to 5 years, or from 30 months to 5 years).

In some examples, the device is produced by additive manufacturing (e.g., 3D printing).

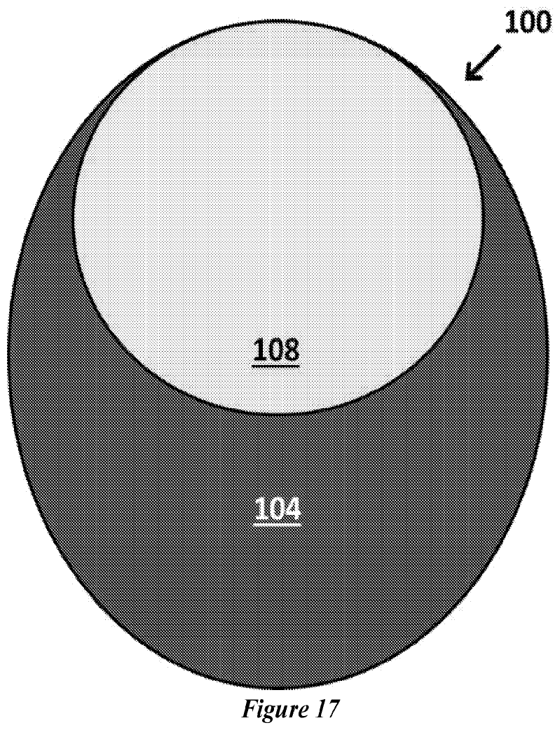

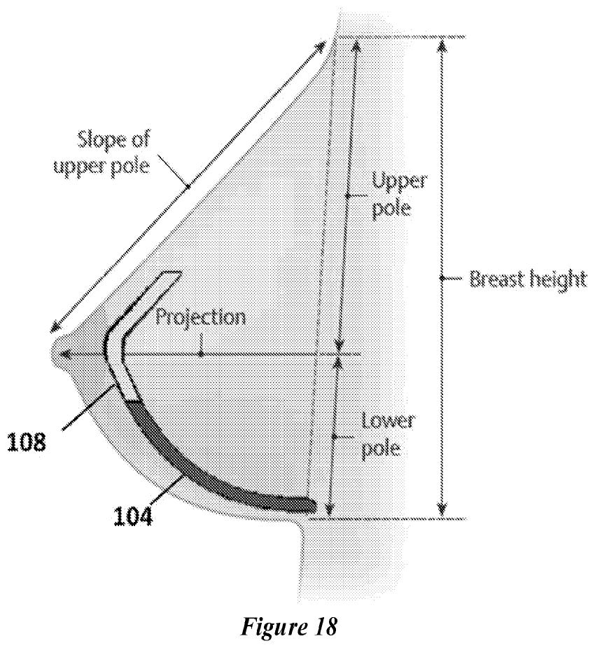

Referring now to FIG. 17 and FIG. 18, in some examples the device 100 comprises a mesh with a plurality of zones, such as a support zone 104 and an elastic zone 108. The support zone 104 is elastic along one direction. The elastic zone 108 has a high amount of elasticity in multiple directions.

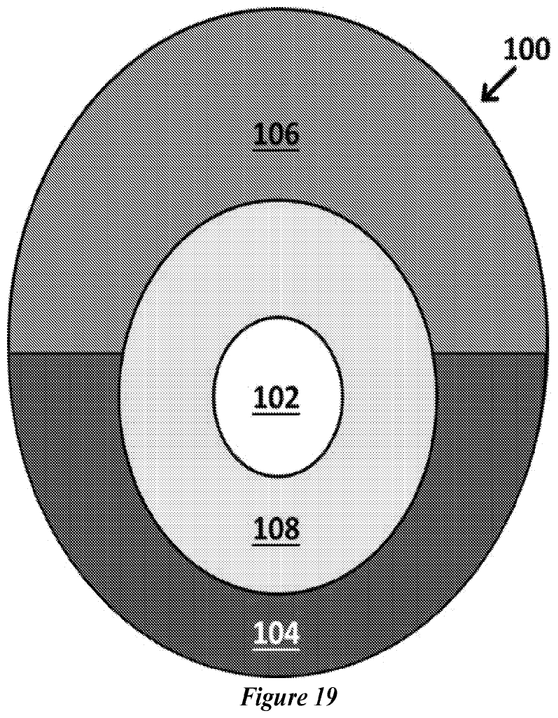

Referring now to FIG. 19, in some examples the device 100 comprises a mesh with a plurality of zones, such as a fat grafting zone 102, a support zone 104, a transition zone 106, and an elastic zone 108. The fat grafting zone 102 has little to no elasticity. The support zone 104 is elastic along one direction. The elastic zone 108 has a high amount of elasticity in multiple directions. The transition zone 106 is more elastic than the support zone 104 and less elastic than the elastic zone 108.

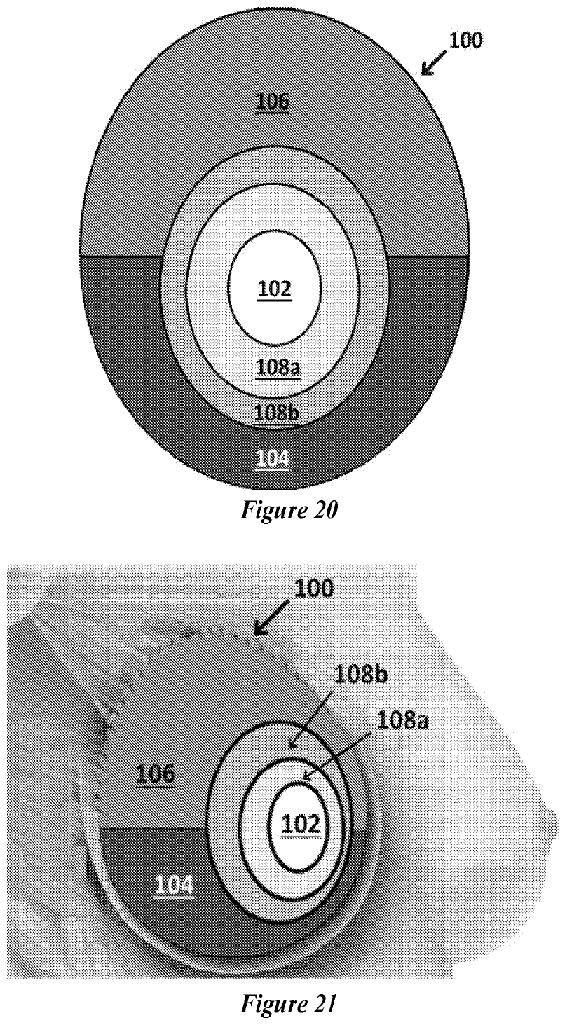

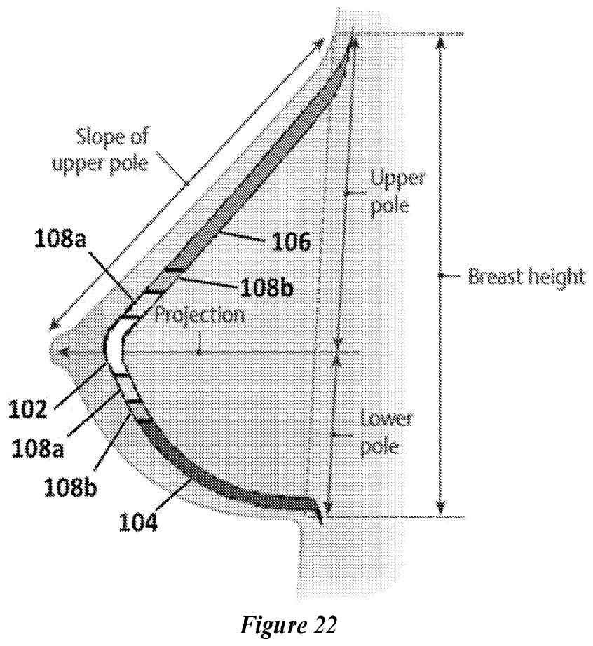

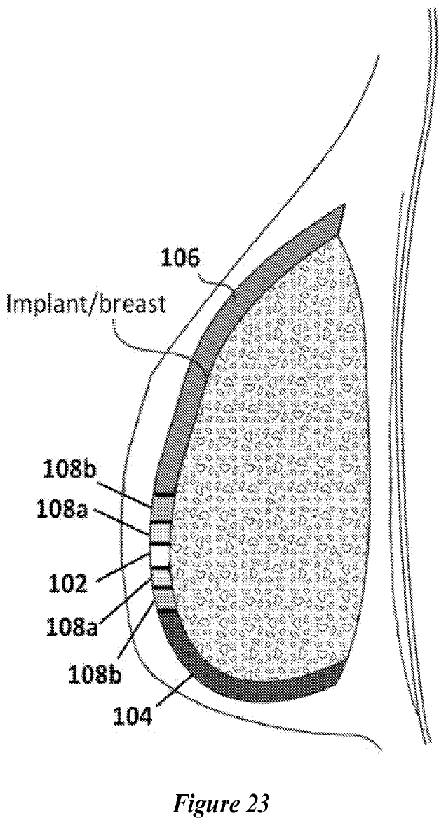

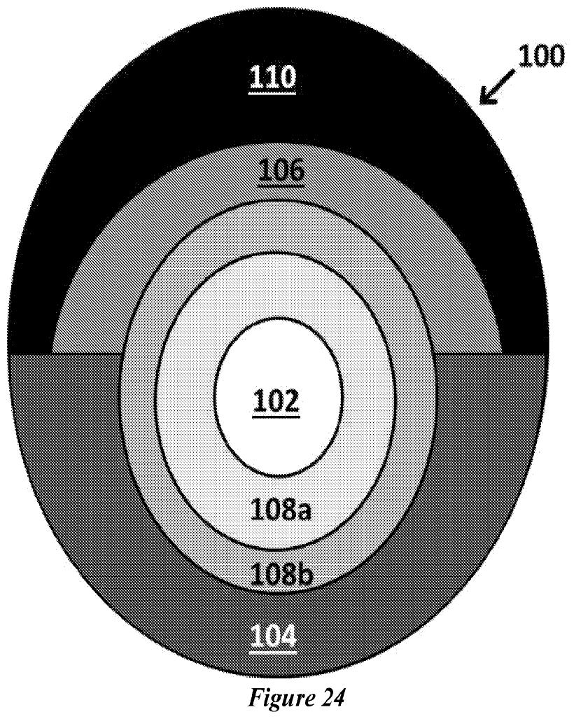

Referring now to FIG. 20-FIG. 23, in some examples the device 100 can comprise a first elastic zone 108a and a second elastic zone 108b, the first elastic zone 108a being more elastic than the second elastic zone 108b.

Referring now to FIG. 21, in some examples, when in use, the device 100 can be anchored to the chest wall of the subject, for example by securing (e.g., suturing) at least a portion of the device 100 to the chest wall of the subject.

Referring now to FIG. 24, in some examples the device 100 can further comprise a skin defect zone 110, the skin defect zone 110 being aligned with skin-growth promoting stroma such that the skin defect zone 110 facilitates epithelization and skin coverage when implanted in the subject.

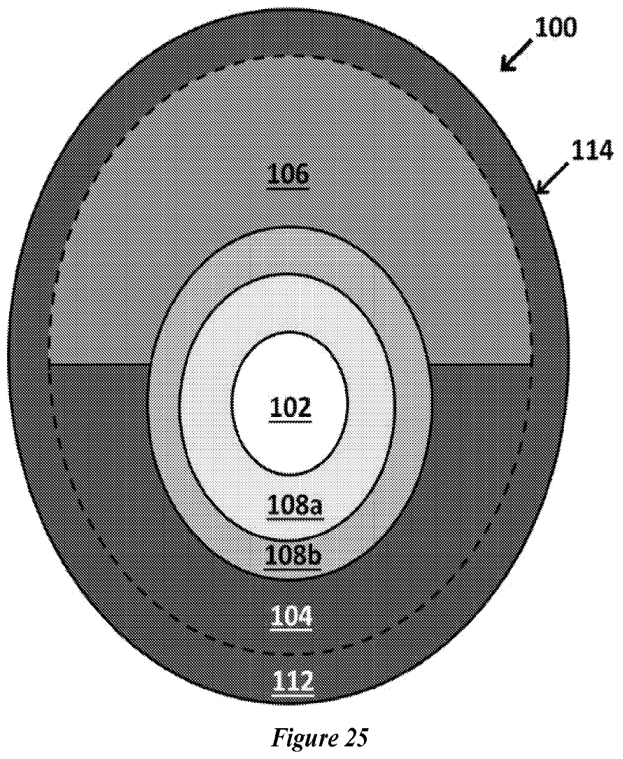

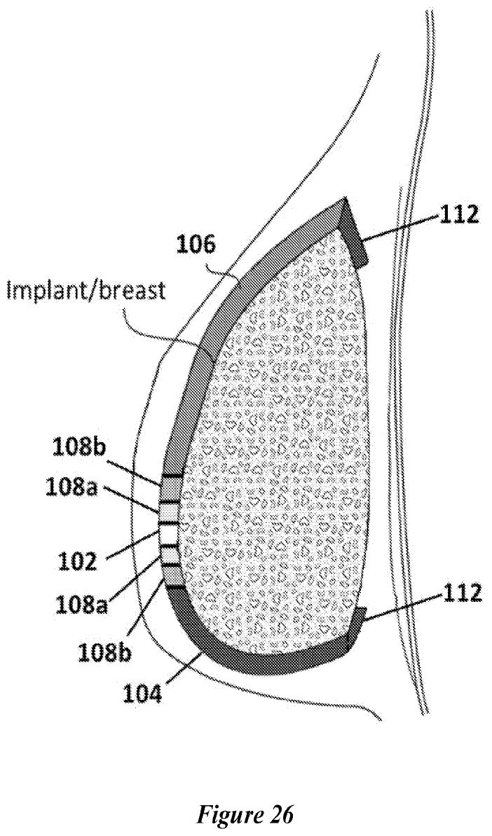

Referring now to FIG. 25 and FIG. 26, in some examples the device 100 can further comprise a cuff 112 extending from at least a portion of the periphery 114 of the device 100. In some examples, the cuff 112 can comprise the same composition and/or pattern as the support zone 104. In some examples, the cuff 112 can comprise a substantially non-elastic material. The cuff 112 can, for example, lay flat or be folded under the device 100 when inserted in the subject. In some examples, the cuff 112 can provide a surface area for contact with the chest wall when inserted in the subject. For example, the cuff 112 can allow for anchoring the device to the chest wall of the subject, for example by securing (e.g., suturing) at least a portion of the cuff 112 to the chest wall of the subject.

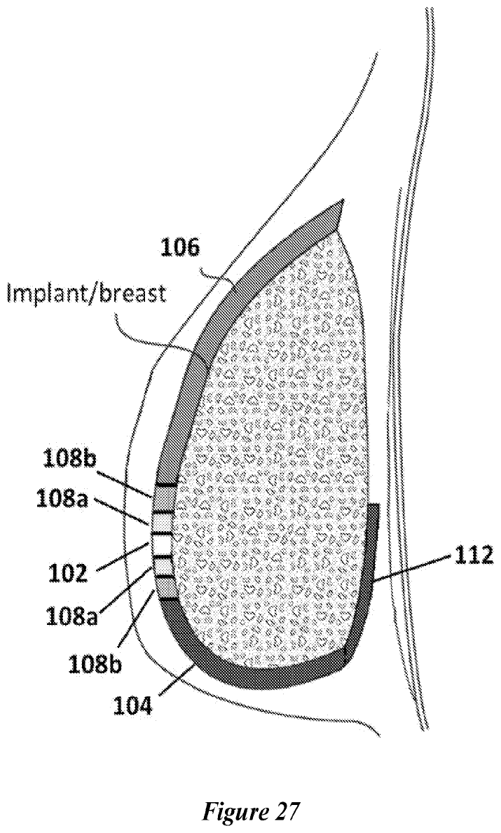

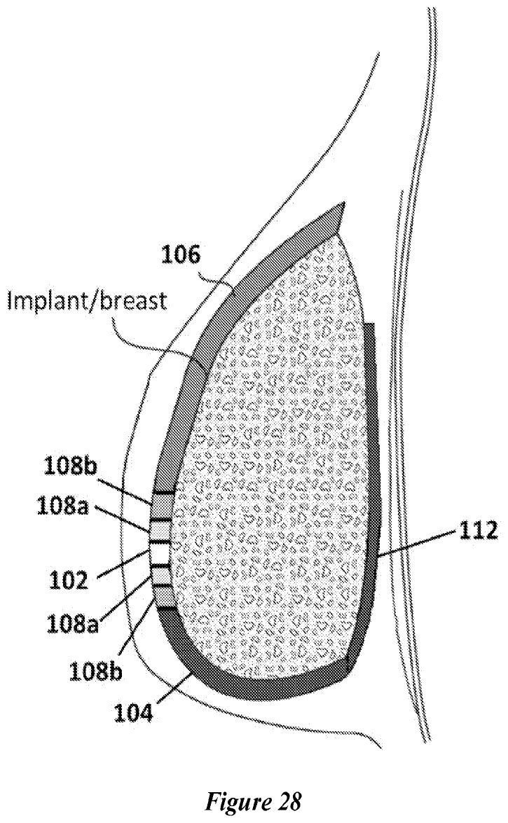

Referring now to FIG. 27 and FIG. 28, in some examples the cuff 112 can form a pocket.

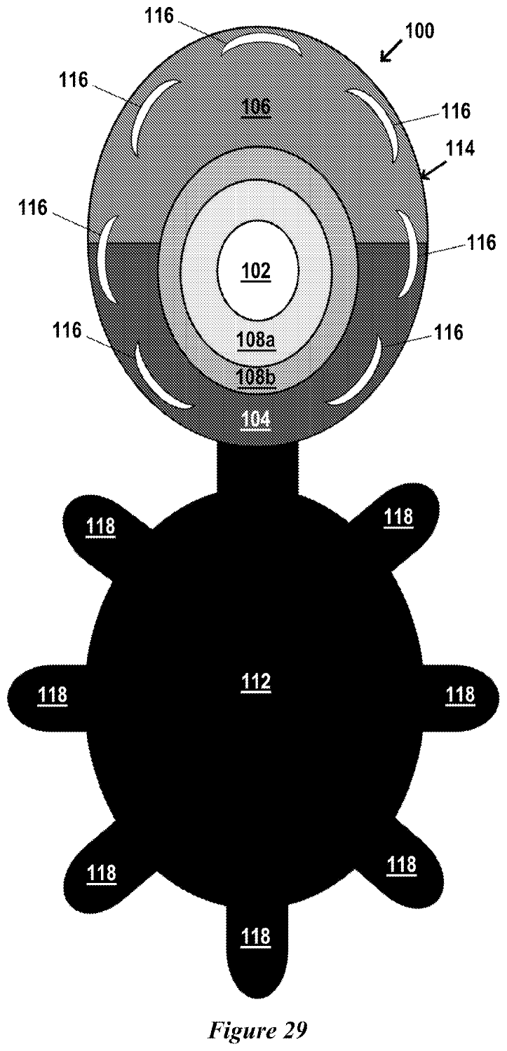

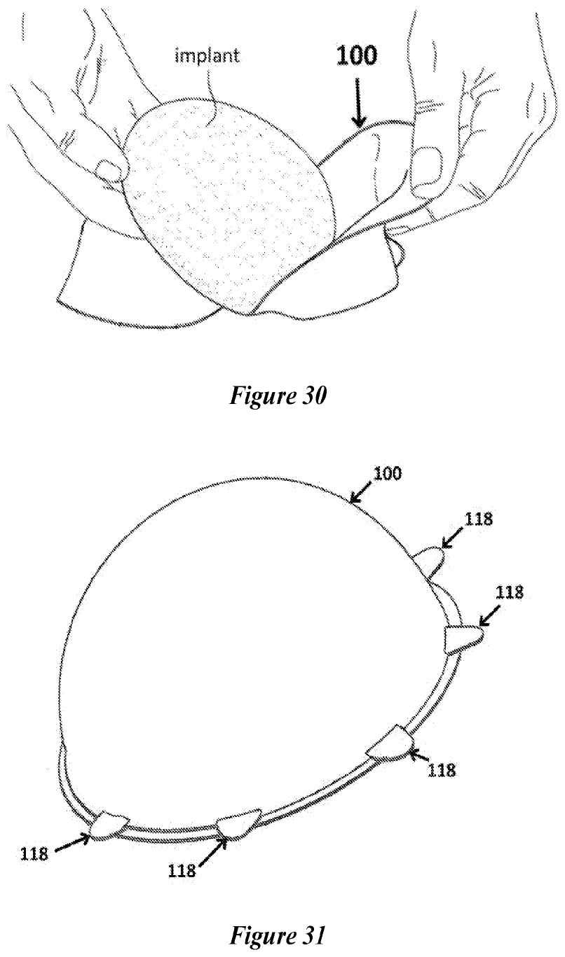

Referring now to FIG. 29-FIG. 31, in some examples, the device 100 can include one or more slots 116 around the periphery 114, and the cuff 112 can include one or more tabs 118 configured to be inserted into the slots 116 to thereby form a pocket. In some examples, the implant can be placed into the pocket, for example as shown in FIG. 30.

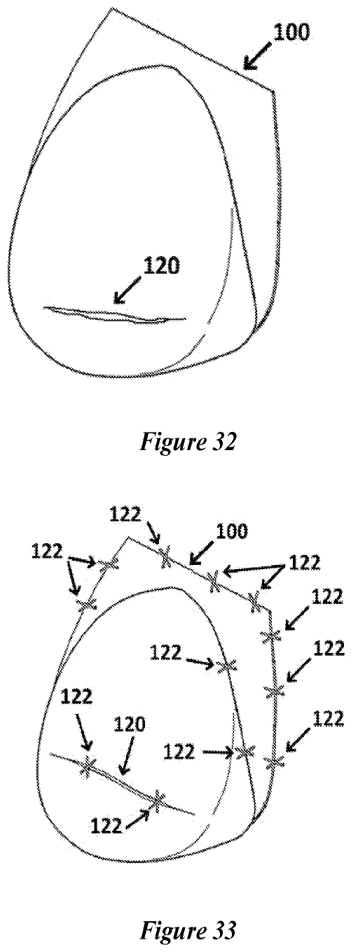

Referring now to FIG. 32-FIG. 33, in some examples, the device 100 can include a slit 120 configured to receive the implant after the pocket is formed. The slit can have a width selected in view of the size of the implant. The slit 120 can, for example, can be 5 centimeters wide to accommodate receiving the implant. In some examples, the device 100 and/or the slit 120 can be secured, for example using sutures 122.

In some examples, the device 100 including the pocket can have an overall teardrop or egg shape, where the cuff portion of the pocket is flat and non-stretchable.

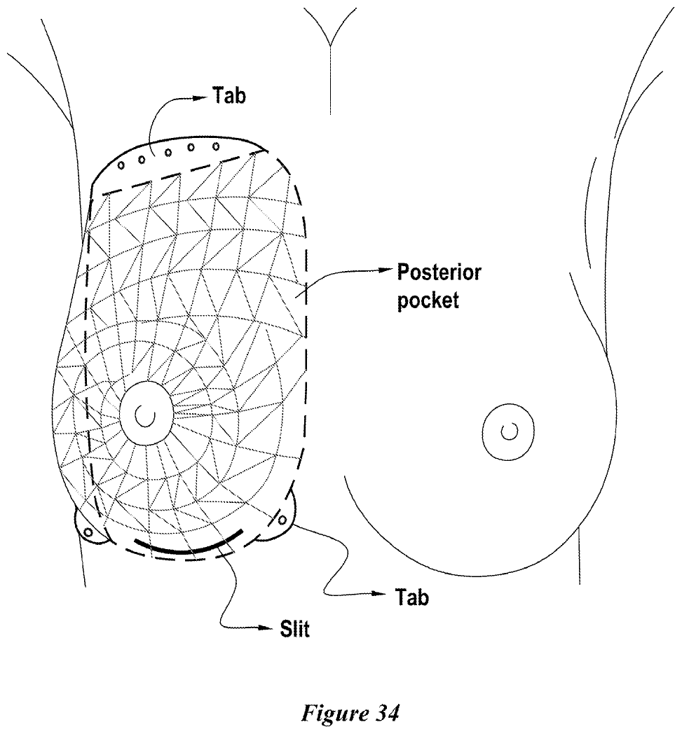

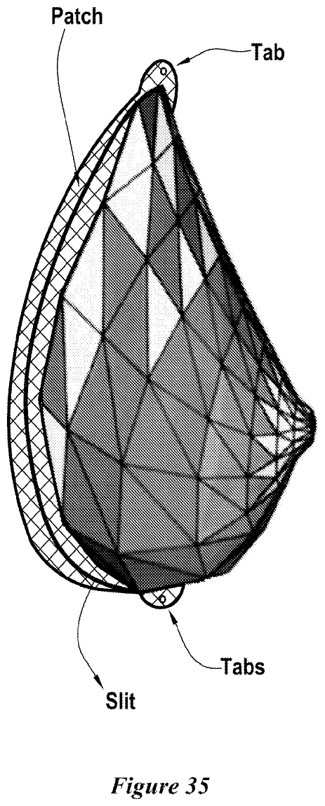

Referring now to FIG. 34-FIG. 35, in some examples, the device can include one or more tabs that can lay flat and provide a surface for anchoring the device to the subject, for example by securing (e.g. suturing) the tabs to the chest wall of the subject. In some examples, the device can include a posterior patch such that the device can form a pocket. In some examples, the device can include a slit configured to receive the implant.

In some examples, the pocket can be specifically designed for the shape of the patient and the shape of the implant inserted into it as a means of support.

In some examples, the device can minimize the subject's foreign body response to the implant.

In some examples, the device can be simply sutured into the subject, for example into a mastectomy or lumpectomy defect, and the implant inserted into it as a means of support and/or minimize foreign body response to the implant.

In some examples, the plurality of zones are specifically designed to optimize the device for long term chape support of the implant and/or to minimize gravitational effects on the tissue.

In some examples, the device has a surface area which can be selected in view of the anatomical location and dimensions thereof. For example, the surface area of the device can be selected to be comparable to the surface area of the anatomical location where the device is to be inserted. For example, the device and the anatomical location can each have an average lateral dimension, and the average lateral dimension of the device can be selected to be comparable to the average lateral dimension of the anatomical location where the device is to be inserted.

When the device is used to support at least a portion of a breast or breast implant, the surface area of the device can be selected in view of the surface area of said portion of the breast or breast implant. For example, if the surface area of the device is too large relative to the portion of the breast or breast implant, the device will be too loose and can lead to undesirable rippling. Alternatively, if the surface area of the device is, for example, too small relative to the portion of the breast or breast implant, the device will be too tight, which can lead to undesirable effects.

### Methods

Also disclosed herein are methods of manufacturing any of the devices 100 disclosed herein. For example, the methods can comprise making the device using additive manufacturing (e.g., 3D printing).

In some examples, the method comprises making the device based on a 3D model. In some examples, the 3D model is based on the Fibonacci equation.

In some examples, the 3D model is based on an anatomical image of a subject. In some examples, the method further comprises collecting the anatomical image of the subject.

Also disclosed herein are methods of treating a subject in need thereof, the methods comprising implanting the device into the subject.

In some examples, the device is implanted into at least a portion breast of the subject. For example, the method can comprise breast reconstruction or augmentation (e.g., full or partial breast reconstruction or augmentation), for example after a lumpectomy.

In some examples, the device is implanted into a breast of the subject. For example, the method can comprise breast reconstruction or augmentation (e.g., full or partial breast reconstruction or augmentation), for example after a lumpectomy or mastectomy.

In some examples, the method further comprises anatomically designing the device for the subject.

In some examples, the methods can comprise breast reconstruction (e.g., full or partial breast reconstruction) and treatment of an oncological disorder, such as breast cancer. In some examples, the devices can further include a therapeutic agent, for example for treatment of the oncological disorder.

For the treatment of oncological disorders, the devices disclosed herein can be administered to a patient in need of treatment in combination with other antitumor or anti-cancer substances and/or with radiation and/or photodynamic therapy and/or with surgical treatment to remove a tumor. These other substances or treatments can be given at the same as or at different times from the devices disclosed herein. For example, the devices disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1—3D Matrix Mesh Printing

Currently, breast implants made out of silicone are traditionally used, but recently received a black box warning by the FDA, due to risk of implant failure, a rare form of ALCL (anaplastic large-cell lymphoma) that is associated with some textured implants, and Breast implant illness. Because of the association of shaped and/or textured implants with the above complications, only round smooth implants are currently approved for clinical use. Therefore, additional means are needed to help support the tissue and/or implant as well as adjust the shape of the spherical implant (e.g., by strategic compression and/or elasticity) to provide a more natural tear drop breast shape.

Current solutions for providing support to an underlying organ and/or implant include ADM (acellular dermal matrix) and fabric/2D mesh structures, all of which suffer from various limitations.

Acellular Dermal Matrix (ADM), a type of surgical mesh, is developed from skin, in which the cells are removed and the support structure is left in place. ADMs vary significantly in their source, processing, level of sterility, biomechanical properties, thickness, final product state, and preparation methods prior to clinical application.

ADMs have, for example, traditionally been used for control of mastectomy pocket for creating a footprint for placement of implant; long term support of the implant and/or prevention of capsular contracture by mitigating the inflammatory reaction that occurs due to implant surface. Further, shaped ADM can improve the breast shape to be more natural (tear drop) when using round implant devices. Currently, surgeons are limited with pre-determined ADM sizes, resulting the inability to match the proper breast size to the patient. Also the ADM material is cadaveric which is expensive and has a limited supply. Moreover, the FDA has not cleared or approved any ADM product for use in breast reconstruction and has further issues statements that certain acellular dermal matrix (ADM) products used in implant-based breast reconstruction may have a higher chance for complications or problems.

Currently there are some companies working in this space to create a replacement for ADM with a 2D mesh. These 2D mesh products are almost like fabric and lack the ability to conform to a convex implant surface and further lack any control or differentiation in the elasticity of the mesh for different zones of the breast.

The technology described herein relates to a 3D Matrix Mesh, which is a 3D printed soft tissue scaffold designed to recreate a natural breast shape that restores support for the overlying mastectomy skin and can, for example, have auxetic properties for differential isotropic and anisotropic control. To optimize soft tissue support, certain parts of the mesh would expand for projection and other parts of the mesh would have no expansion for improved support. Further, the devices described herein can rely on compositions such as bioresorbable polymers, collagen, bioinks, etc.

Combining both a computational breast analysis model with the 3D Matrix Mesh printing technology would have the ability to mimic a natural breast shape customized to the patient.

An advantage of the 3D Matrix Mesh would be the ability to customize to the patient's natural breast shape. Currently, surgeons are limited with pre-determined ADM sizes, resulting the inability to match the proper breast size to the patient. Also the ADM material is cadaveric which is expensive and has a limited supply. Utilizing a computational breast analysis model to match the patient by scanning their natural breast shape permits a customized designed for breast reconstruction.

The 3D Matrix Mesh can contain the following materials to create a self-regenerating and biodegradable device: 1. Bioresorbable Polymer; 2. Recombinant Human Collagen; and 3. Adipose Cellular Stromal Matrix. In some examples, the 3D matrix mesh can include a bioink.

The 3D Matrix Mesh can be structured to support the soft breast tissue. In addition, the mesh can provide support for a traditional implant or other implantable devices. The matrix mesh can have different properties in various locations of the breast envelope to provide support and encourage fat-in growth in specific breast regions. For example, the lower part of the breast, which supports the weight of the implant, can have a tighter, less elastic pattern of the mesh, whereas over the top part of the breast above the nipple area, the mesh can have a more stretching design to allow maximum projection of the underlining implant and to improve breast shape.

At the present time, surgeons do not have the ability to replicate the original breast shape of a patient with the current ADM products or current mesh products. However, combining the computational breast analysis model with the 3D Matrix Mesh technology would revolutionize breast reconstruction and customize the patients' anatomical breast size maintaining a natural breast complementing the original breast shape.

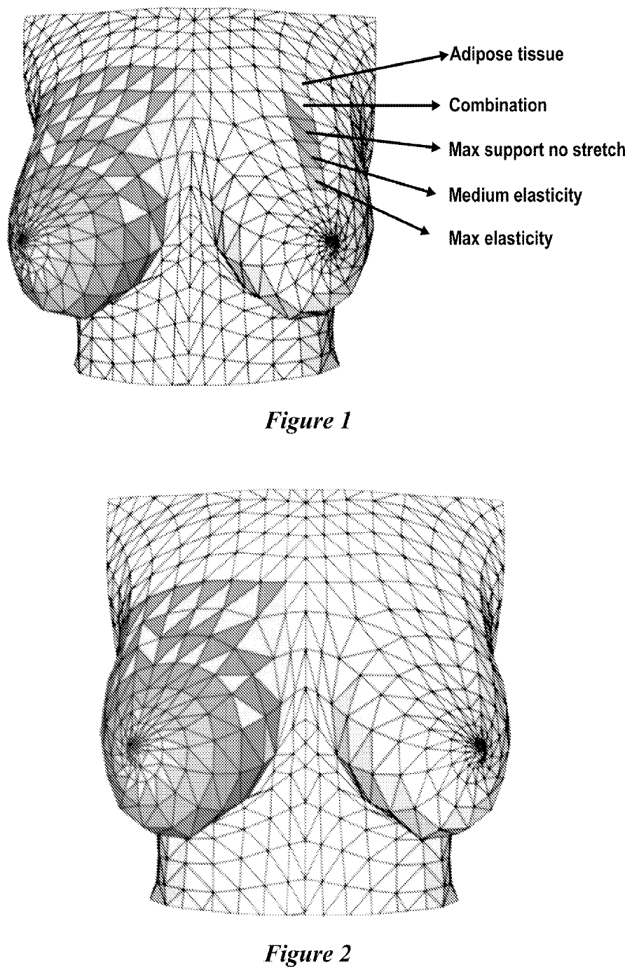

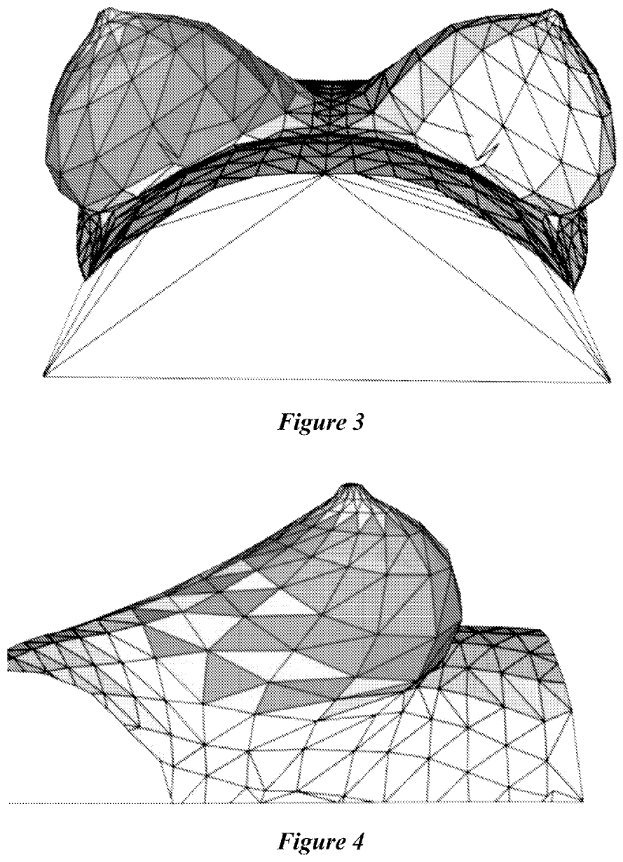

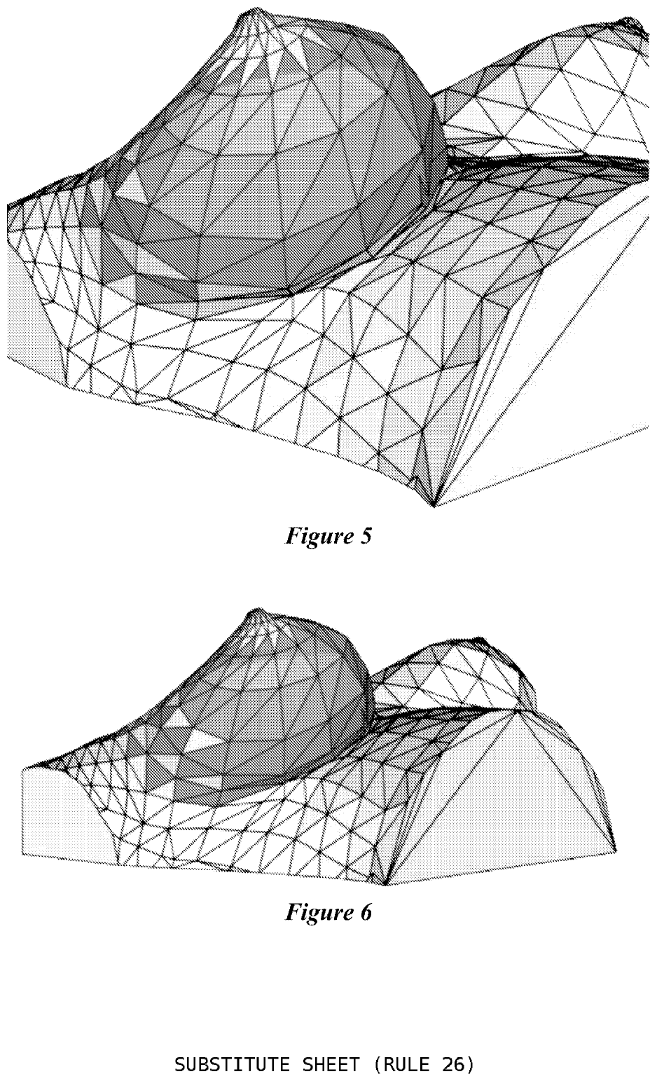

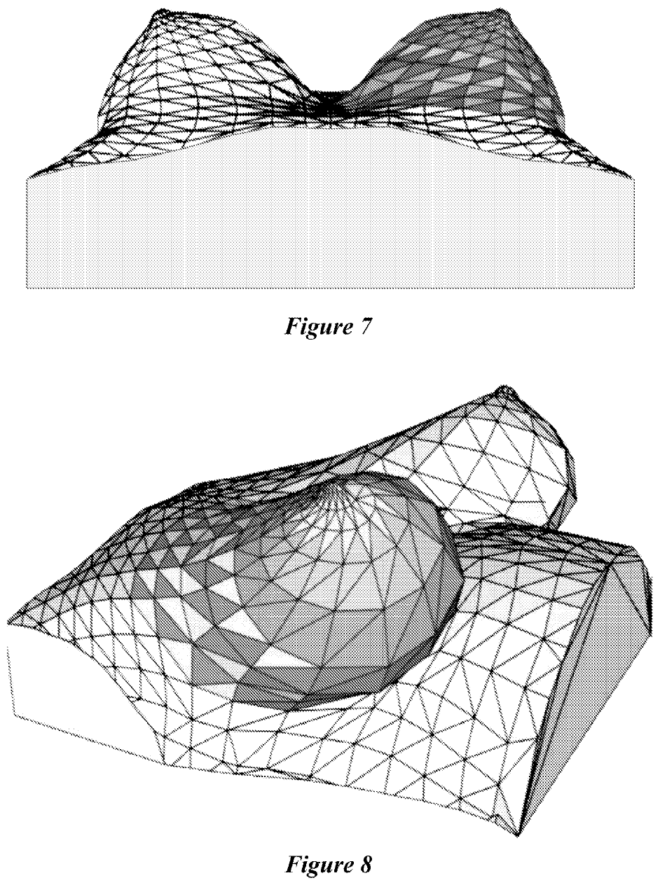

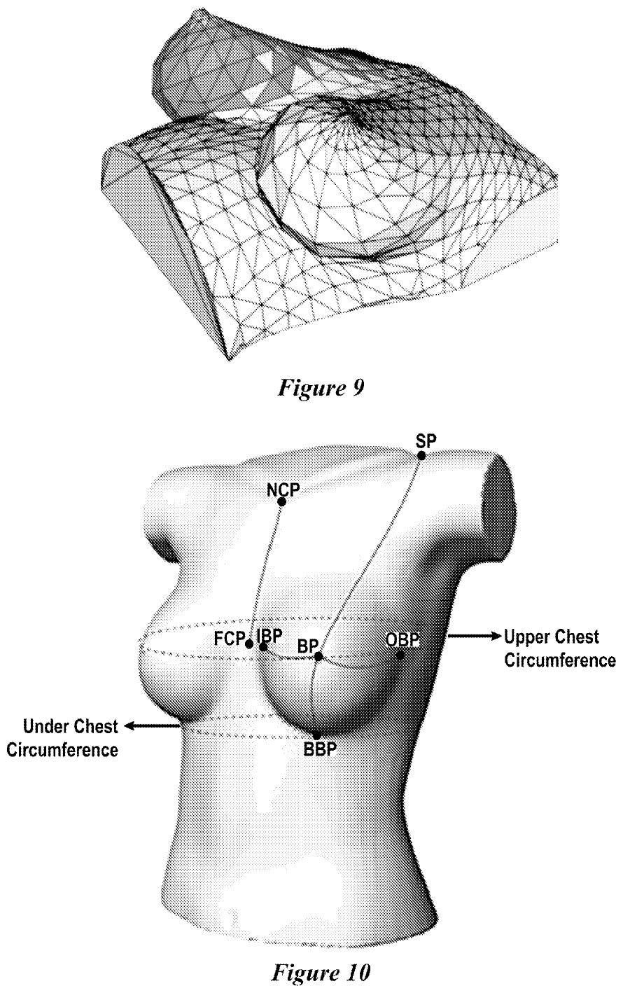

As shown in the FIG. 1-FIG. 9, the matrix mesh can be a 3D printed construct with various patterns at specific locations that can provide isotropic and anisotropic breast support. Due to the ability to be printed as a 3D structure, the mesh can be customized to the specific size and anatomy of the patient. The 3D Bio Matrix Mesh can be used with various types and sizes of implants. The 3D Matrix Mesh can combine recombinant human collagen, bioresorbable polymer, and adipose cellular stromal matrix materials that promote fat in growth.

This can allow the Matrix Mesh to have better inherited qualitative properties, including, for example, to support soft breast tissue; to provide support for traditional implants and other implantable devices; and to take advantage of the ability to control 3D printing properties for different material property location that can support and encourage isolated fat-in growth to specific regions of the breast.

By specifically designing the printing pattern at various parts of the breast, the amount of elasticity can be controlled. As such, the lower part of the breast, which supports the weight of the implant, can have a tighter less elastic pattern of the mesh. The mesh in the area surrounding the nipple areola complex can have the most elasticity to allow maximum projection of the underlying implant and to improve breast shape.

For example, the 3D Matrix Mesh can be designed and shaped according to the patient's structural needs as shown, for example, in FIG. 1-FIG. 10. To optimize breast support, a less elastic mesh pattern ring would extend from the bottom of the breast to the superior pectoral fascia where it would be anchored by sutures, thereby forming a suspension effect for the underlying implant. Prior to surgery, a 3D computational breast model can be obtained to customize the patient's breast via scan, and the 3D Matrix Mesh would be printed for the patient. Additionally, a variety of predetermined sizes can be available if a scan is not possible.

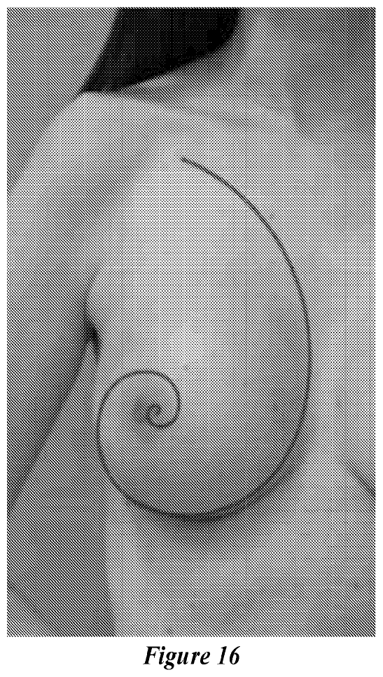

To accomplish a natural breast shape, the 3D Matrix Mesh can be printed into a similar proportion to the Fibonacci sequence (FIG. 16) and an auxetic structure maintaining the integrity of the patient's natural geometric breast shape.

### Microscopic Characteristics

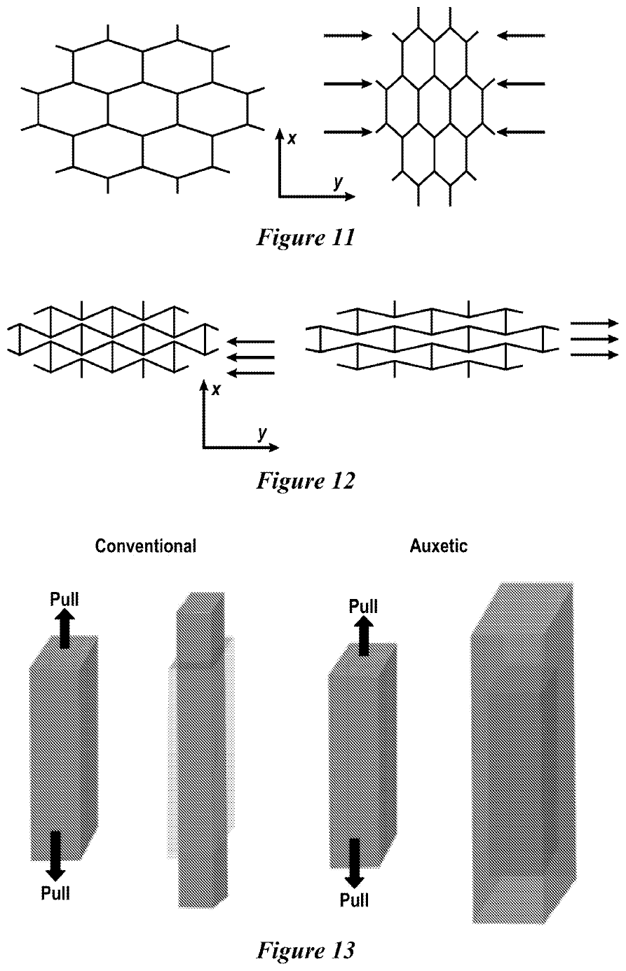

3D Printing Pattern. Auxetic materials have specific properties when compared to non-auxetic materials based on their structure and deformation behavior (FIG. 11-FIG. 13). Unlike traditional materials that elongate when stretched, auxetic materials and patterns expand laterally when stretched longitudinally.

Poisson ratio (v)=lateral contractile strain/longitudinal tensile strain

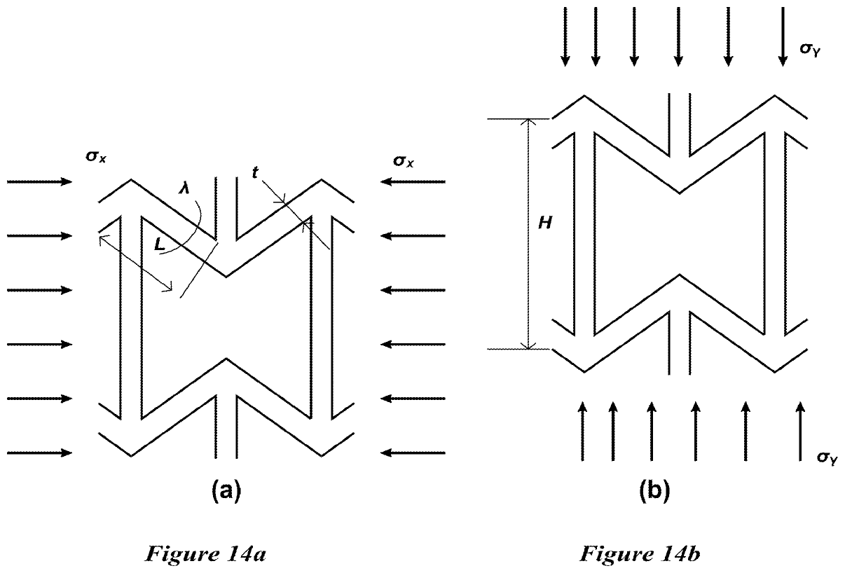

Pattern 1: Honeycomb hexagonal pattern—Positive v ratio: cells elongate along the y axis and close up along the x axis when stretched along the y axis (FIG. 11, FIG. 13).

Pattern 2 Modified Reentrant honeycomb pattern—Negative v ratio: cells undergo elongation in both x and y axis when stretched (FIG. 12, FIG. 13).

Nonauxetic and auxetic structure. The auxetic portion of the mesh can be printed around the nipple because there is stretching one direction, but also elastically in the second direction (FIG. 13). As it is stretched it is not contracting in the perpendicular direction, which is also stretching, especially in the area where there is more projection needed (FIG. 13). The non-auxetic portion would be 3D printed to provide support superior to the inframammary fold (FIG. 13).

Auxetic and nonauxetic materials. The material used can also be adjusted to be auxetic or non-auxetic. For example, an auxetic material printed in an auxetic pattern can amplify the auxetic properties, which can be exploited to maximize control of the support and/or shape of the underlying implant/organ.

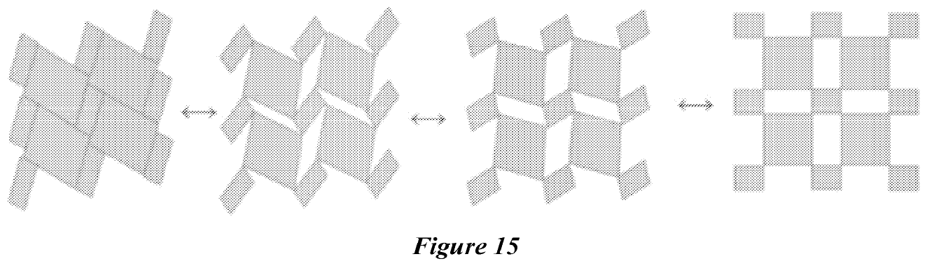

Isotropic and Anisotropic Material. The material used can also be adjusted to be isotropic or anisotropic, allowing an additional degree of control of the stretch/support in the different zones of the mesh (FIG. 14a-FIG. 15). The properties of an isotropic material are identical in all directions. Meanwhile, the properties of an anisotropic depend on/vary based on the direction.

3D Printing Zones. The mesh can include different zones, such as, for example, those illustrated in FIG. 1-FIG. 9. Referring now to FIG. 1-FIG. 9, the mesh can include a Fat Grafting Zone (Solid yellow), which can maximize adipose tissue and has little to no elasticity. The mesh can further, for example, include a support zone (pattern 1, purple), which can maximize support (purple) and stretch in one direction (e.g., non-auxetic honeycomb), such that when elongated horizontally, vertical shortening occurs, maximizing breast support. The mesh can further include an Elastic Zone (Pattern 2, light pink), which can maximize projection (light pink) and can be auxetic, allowing elongation in both directions. The mesh can further include a Transition zone (Mix of Pattern 1 & 2, mixed yellow and purple), which can transition between a zone of support and a zone of maximal projection.

The materials used to maximize properties of each zone. The materials used to print each zone can be selected based on maximizing the properties of each zone. For example, the materials used to print each zone can be selected based on the properties of the following materials: a. Bioresorbable Polymer; b. Recombinant Human Collagen; and c. Adipose Cellular Stromal Matrix.

The mesh can, in some examples, include an additional feature for cases where there is a skin defect. For these cases, there can also be a solid component of the mesh that can be aligned with skin growth-promoting stroma to facilitate epithelization and skin coverage. There is potential to customize a solid patch of the matrix for skin replacement option, which is aligned with skin keratinocyte promoting factors for skin epithelization.

Macroscopic characteristics. Macroscopic characteristics of the zone include the location of the different zones and proportion of the entire construct (FIG. 16).

With the current ADM technology, surgeons are not able to replicate a natural-looking breast compared to an original breast shape. However, with the computational breast analysis model combined with the 3D Matrix Mesh technology, surgeons can reconstruct a breast to complement the original breast shape surgically. In addition, the 3D Matrix Mesh cellular make-up can allow faster healing and cellular regeneration to support fat-in growth.

The 3D Matrix Mesh can allow customization and allow surgeons to control the implant position and prevent the need for submuscular implant placement.

The Matrix Mesh can have better inherited qualitative qualities, such as support of soft breast tissue, support for traditional implants and other implantable devices, and the ability to control 3D printing properties for different material property location that can support and encouraging isolated fat-in growth to specific regions of the breast. As such, at the lower part of the breast, which supports the weight of the implant, the mesh can have a tighter less elastic pattern, but over the top part of the breast above the nipple area, the mesh can have a more stretchable design to allow maximum projection of the underlining implant and to improve breast shape.

Incorporating computational breast analysis model combined with the 3D Matrix Mesh technology can incorporate a more natural breast when compared to the original breast shape for the patient. In addition to providing mechanical stability in the breast, the 3D Matrix Mesh can facilitate cellular wound healing and fat-growth regeneration.

The technology can be extended to other medical field that would need a customized 3D Matrix mesh related surgery.

### Example 2

The technology disclosed herein envisions replacing the ADM using a computer breast analysis model to create a 3D matrix printed mesh customized to the patient's anatomical breast shape. Also, allowing the mesh material to be customized to support the implant's weight and allow more elasticity in areas that are needed while providing support for the underlying implant and finally, it would contain materials that can promote and encourage isolated fat-in growth. The 3D Matrix Mesh can promote fat-in growth regeneration and quicker healing time.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

What is claimed is:

1. A device configured to be inserted into an anatomical location of a subject, the device comprising a mesh with a plurality of zones having different elastic properties, each zone having a composition and a pattern, wherein the different elastic properties are based on the composition, the pattern, or combination thereof.

2. The device of claim 1, wherein the composition of each of the plurality of zones comprises an isotropic material, an anisotropic material, an auxetic material, a non-auxetic material, or a combination thereof.

3. The device of claim 1 or claim 2, wherein the pattern of each of the plurality of zones comprises an auxetic pattern, a non-auxetic pattern, or a combination thereof.

4. The device of any one of claims 1-3, wherein the mesh comprises a fat grafting zone having little to no elasticity.

5. The device of claim 4, wherein the fat grafting zone comprises adipose tissue.

6. The device of claim 5, wherein the adipose tissue comprises autologous adipose tissue.

7. The device of any one of claims 1-6, wherein the mesh comprises a support zone, the support zone being elastic along one direction.

8. The device of claim 7, wherein the support zone has a non-auxetic pattern.

9. The device of claim 7 or claim 8, wherein the support zone comprises an anisotropic material, a non-auxetic material, or a combination thereof.

10. The device of any one of claims 1-9, wherein the mesh comprises an elastic zone, the elastic zone having a high amount of elasticity in multiple directions.

11. The device of claim 10, wherein the elastic zone has an auxetic pattern.

12. The device of claim 10 or claim 11, wherein the elastic zone comprises an isotropic material, an auxetic material, or a combination thereof.

13. The device of any one of claims 1-12, wherein the mesh comprises a transition zone, the transition zone being more elastic than the support zone and less elastic than the elastic zone.

14. The device of any one of claims 1-13, wherein the mesh comprises a skin defect zone, the skin defect zone being aligned with skin-growth promoting stroma such that the skin defect zone facilitates epithelization and skin coverage when implanted in the subject.

15. The device of any one of claims 1-14, wherein the plurality of zones each have a different composition, a different pattern, or a combination thereof.

16. The device of any one of claims 1-15, wherein the plurality of zones have the same composition and different patterns, such that the different properties are based on the pattern of each zone.

17. The device of any one of claims 1-16, wherein the plurality of zones each have a different composition and a different pattern.

18. The device of any one of claims 1-17, wherein the composition of each of the plurality of zones independently comprises a polymer such as a bioresorbable polymer, collagen, adipose cellular stromal matrix, adipose tissue, or a combination thereof.

19. The device of any one of claims 1-18, wherein one or more of the plurality of zones independently is porous.

20. The device of any one of claims 1-19, wherein the composition of one or more of the plurality of zones independently comprises poly(ethylene glycol) diacrylate (PEGDA), poly(ethylene glycol) dimethacrylate (PEGDMA), poly(ethylene glycol) diacrylamide (PEGDAAm), gelatin methacrylate (GelMA), collagen methacrylate, silk methacrylate, hyaluronic acid methacrylate, chondroitin sulfate methacrylate, elastin methacrylate, cellulose acrylate, dextran methacrylate, heparin methacrylate, NIPAAm methacrylate, Chitosan methacrylate, polyethylene glycol norbornene, polyethylene glycol dithiol, thiolated gelatin, thiolated chitosan, thiolated silk, silk, PEG based peptide conjugates, cell-adhesive poly(ethylene glycol), MMP-sensitive poly(ethylene glycol), PEGylated fibrinogen, aliphatic poly-isocyanate, poly-aliphatic isocyanates, poly-4-hudroxybutyrate, poly(l-lactide) (PLLA), bioceramic particles, L-lactide (LLA), sub-dermal explant comprising polycaprolactone (PCL), polyurethane, poly(D) lactide, poly(lactic-co-glycolic) acid, poly(α-hydroxy acids), cross-linked polyester hydrogels, poly(orthoesters), polyanhydrides, or a combination thereof.

21. The device of any one of claims 1-20, wherein the composition of one or more of the plurality of zones independently comprises aliphatic poly-isocyanate, poly-aliphatic isocyanates, poly-4-hudroxybutyrate, poly(l-lactide) (PLLA), bioceramic particles, L-lactide (LLA), sub-dermal explant comprising polycaprolactone (PCL), polyurethane, poly(D) lactide, poly(lactic-co-glycolic) acid, poly(α-hydroxy acids), cross-linked polyester hydrogels, poly(orthoesters), polyanhydrides, or a combination thereof.

22. The device of any one of claims 1-21, wherein the composition of one or more of the plurality of zones independently comprises a polyester, such as poly(glycerol-dodecanoate) (PGD).

23. The device of any one of claims 1-22, wherein the composition of one or more of the plurality of zones independently comprises a poly(ether-ester).

24. The device of any one of claims 1-23, wherein the composition of one or more of the plurality of zones independently comprises polydioxanone (PDO).

25. The device of any one of claims 1-24, wherein the composition of each of the plurality of zones comprises polydioxanone (PDO).

26. The device of any one of claims 1-25, wherein the composition of one or more of the plurality of zones independently comprises a polyolefin, such as polypropylene.

27. The device of any one of claims 1-26, wherein the composition of each of the one or more of the plurality of zones independently comprises polyglycolic acid (PGA).

28. The device of any one of claims 1-27, wherein the composition of each of the one or more of the plurality of zones independently comprises a bioink.

29. The device of any one of claims 1-28, wherein the device further comprises a therapeutic agent dispersed within the composition of one or more of the plurality of zones.

30. The device of claim 29, wherein the therapeutic agent is dispersed substantially homogeneously throughout the zone(s) and/or device.

31. The device of claim 29 or claim 30, wherein the therapeutic agent comprises an anticancer agent, anti-inflammatory agent, analgesic agent, antimicrobial agent, or a combination thereof.

32. The device of any one of claims 29-31, the therapeutic agent comprises a chemotherapeutic agent, an immunotherapeutic agent, or a combination thereof.

33. The device of any one of claims 1-32, wherein device is configured to be stable for an amount of time of from 6 weeks to 5 years weeks after the device is implanted in the subject.

34. The device of any one of claims 1-33, wherein the device comprises a support zone and an elastic zone.

35. The device of any one of claims 1-34, wherein the device comprises a fat grafting zone, a support zone, a transition zone, and an elastic zone.

36. The device of any one of claims 1-35, wherein the device comprises a first elastic zone and a second elastic zone, the first elastic zone being more elastic than the second elastic zone.

37. The device of any one of claims 1-36, wherein the device has a periphery and the device further comprises a cuff extending from at least a portion of the periphery of the device.

38. The device of claim 37, wherein the cuff forms a pocket.

39. The device of any one of claims 1-38, wherein the device is configured to support and/or reshape at least a portion of an organ and/or at least a portion of an implant when inserted in the subject.

40. The device of any one of claims 1-39, wherein the device is configured to support and/or reshape an organ and/or an implant when inserted in the subject.

41. The device of any one of claims 1-40, wherein the anatomical location comprises a breast of the subject.

42. The device of claim 41, wherein the device is configured to support at least a portion of a breast or at least a portion of a breast implant.

43. The device of claim 41 or claim 42, wherein the device is configured to support a breast or a breast implant.

44. The device of any one of claims 1-43, wherein device is formed from a model based on a tessellation of polyhedrons.

45. The device of any one of claims 1-44, wherein the device is formed from a computational 3D space-filling model.

46. The device of any one of claims 1-45, wherein the device is not flat.

47. The device of any one of claims 1-46, wherein the device has a three dimensional shape.

48. The device of any one of claims 1-47, wherein the device has a three-dimensional parametric teardrop shape.

49. The device of any one of claims 1-48, wherein the device has a two dimensional shape that can stretch to a three dimensional teardrop shape that conforms to support and/or reshape an organ and/or an implant when inserted in the subject.

50. The device of any one of claims 1-49, wherein the device has a three-dimensional parametric teardrop shape following the Fibonacci equation.

51. The device of any one of claims 1-50, wherein the device is anatomically designed for the subject.

52. The device of any one of claims 1-51, wherein the device is produced by additive manufacturing (e.g., 3D printing).

53. The device of any one of claims 1-52, wherein the device is a single piece of mesh (e.g., monolithic).

54. The device of any one of claims 1-53, wherein the device is biocompatible.

55. A method of manufacturing the device of any one of claims 1-54, the method comprising making the device using additive manufacturing (e.g., 3D printing).

56. The method of claim 55, wherein the method comprises making the device based on a 3D model.

57. The method of claim 56, wherein the 3D model is based on the Fibonacci equation.

58. The method of claim 56 or claim 57, wherein the 3D model is based on an anatomical image of a subject.

59. The method of claim 58, wherein the method further comprises collecting the anatomical image of the subject.

60. A method of treating a subject in need thereof, the method comprising implanting the device of any one of claims 1-54 into the subject.

61. The method of claim 60, wherein the device is implanted into at least a portion of a breast of the subject.

62. The method of claim 61, wherein the method comprises breast reconstruction or augmentation (e.g., full or partial breast reconstruction or augmentation), such as after a lumpectomy or mastectomy.

63. The method of claim 60, wherein the device is implanted into a breast of the subject.

64. The method of claim 63, wherein the method comprises breast reconstruction or augmentation.

65. The method of any one of claims 60-64, wherein the method further comprises anatomically designing the device for the subject.
